(19) United States
(12) Patent Application Publication
(10) Pub. No.: US 2022/0211320 A1
(43) Pub. Date: Jul. 7, 2022

(54) COMPOSITE MEDICAL TREATMENT APPARATUS USING SKIN MAP

(71) Applicant: CMLAB CO., LTD., Seoul (KR)

(72) Inventor: Tae Ho Ha, Gyeonggi-do (KR)

(21) Appl. No.: 17/595,439

(22) PCT Filed: May 18, 2020
(86) PCT No.: PCT/KR2020/006492

§ 371 Date: Nov. 17, 2021

(30) Foreign Application Priority Data
May 17, 2019 (KR) 10-2019-0058204

(51) Int. Cl. A61B 5/00; A61N 7/02; A61N 5/06; A61N 5/067; G16H 50/20; G16H 20/40

(52) U.S. Cl. CPC A61B 5/442; A61N 7/02; A61N 5/0616; A61N 5/067; A61N 2007/0034; G16H 50/20; G16H 20/40; A61N 2005/0626; A61B 5/4836

(57) ABSTRACT

Composite medical treatment apparatus using skin map disclosed. The composite medical treatment apparatus includes a handpiece configured for measuring a skin condition associated with a measurement position on a region of interest in a skin map generation step, and configured for measuring a treatment position on the region of interest and applying a treatment medium according to a control parameter corresponding to the treatment position in a treatment step and a main controller configured for associating the measurement position with the skin condition in the skin map generation step, and configured for retrieving the skin condition associated with the measurement position based on the treatment position to generate the control parameter in the treatment step.

### FIELD

The present invention relates to a composite medical treatment apparatus.

### BACKGROUND

Compared with surgical operation, the popularity of non-incisional treatment, which has low risk of bleeding, infection, and scarring and allows daily life immediately after treatment, is increasing in recent years. In the non-incisional treatment, for example, a laser, an ultrasound and so on are applied to the skin for treatment.

High Intensity Focused Ultrasound (HIFU) is mainly used for non-invasive treatment. HIFU can selectively coagulate, necrotize and kill lesions. In particular, if HIFU is used, non-invasive/non-anesthetic treatment becomes possible, and accurate and repeated treatment of lesions becomes possible. For this reason, it is used in various fields such as tumor treatment (uterine fibroids, liver cancer, breast cancer, etc.), fat and wrinkle removal.

Lasers are mainly used to remove lesions by removing tissue or evaporating tissue in place of surgical knives. It is used in various fields such as dermatology, otorhinolaryngology, ophthalmology, and surgery in that microsurgery is possible without bleeding and there is little pain and scarring.

HIFU enables treatment by non-invasively applying energy to the inside of the skin (dermis, subcutaneous fat), but may cause side effects when treated near the skin surface (epidermis). On the other hand, although laser is difficult to deliver energy to the inside of the skin, it is excellent at applying energy to the epidermis. For this reason, various treatments for skin diseases and cosmetic treatment using laser have been developed. Therefore, it is necessary to develop a treatment device capable of simultaneously performing ultrasound and laser treatment in that the treatment effect can be improved when ultrasound and laser are combined.

### SUMMARY

The present invention is to provide a composite medical treatment apparatus capable of performing composite treatment using high-intensity focused ultrasound and laser according to a patient's skin condition.

According to one aspect of the present invention, a composite medical treatment apparatus using skin map is provided. The composite medical treatment apparatus may include a handpiece configured for measuring a skin condition associated with a measurement position on a region of interest in a skin map generation step, and configured for measuring a treatment position on the region of interest and applying a treatment medium according to a control parameter corresponding to the treatment position in a treatment step and a main controller configured for associating the measurement position with the skin condition in the skin map generation step, and configured for retrieving the skin condition associated with the measurement position based on the treatment position to generate the control parameter in the treatment step.

In one embodiment, the treatment medium is a high-intensity focused ultrasound and a laser, wherein the handpiece may include an ultrasonic transducer configured for generating the high-intensity focused ultrasound and applying to the region of interest, a laser applicator configured for applying the laser generated in the main controller to the region of interest and an optical position sensor configured for generating a measurement position and a treatment position information.

In one embodiment, the skin condition is a skin thickness, and the ultrasound transducer is a dual-mode ultrasound transducer configured for applying a low-intensity ultrasound for measuring the skin thickness to the region of interest.

In one embodiment, the skin condition is a skin elasticity and the handpiece measures the skin elasticity from time to recover an original state after applying a pressure to the skin on the region of interest.

In one embodiment, the handpiece may include a probe being in contact with the skin, a vertical driver configured for applying the pressure to the skin by moving the probe vertically, and removing the pressure when the probe comes into contact with the skin and a sensor configured for measuring a vertical displacement of the probe after the pressure is removed.

In one embodiment, the handpiece may include a HIFU-laser generator configured for generating the high-intensity focused ultrasound and the laser, a vertical driver configured for applying the pressure to the skin by moving the HIFU-laser generator vertically, and removing the pressure when the HIFU-laser generator comes into contact with the skin and a sensor configured for measuring a vertical displacement of the HIFU-laser generator after the pressure is removed.

In one embodiment, the skin condition in the skin map is associated with the measurement position for each point.

In one embodiment, the skin condition in the skin map is associated with a region having the skin condition that is the same or falls within an allowable error range.

In one embodiment, the control parameter is a combination of a selection of the treatment medium to be applied to a treatment point corresponding to the treatment location, an intensity of the selected treatment medium, an output time of the selected medium, and an application depth of the selected medium.

According to another aspect of the present invention, a method of driving a composite medical treatment apparatus using a skin map is provided. The method may include generating a skin map by measuring a skin condition associated with a measurement position on a region of interest using a handpiece, measuring a treatment position on the region of interest using the handpiece, retrieving the skin condition associated with the measurement position corresponding to the treatment position from the skin map and generating a control parameter for controlling an operation of the composite medical treatment apparatus using the retrieved skin condition.

In one embodiment, the generating a skin map by measuring a skin condition associated with a measurement position on a region of interest using a handpiece may include measuring a direction and a distance of movement of the handpiece in contact with the region of interest, measuring the skin condition of the region of interest while moving the handpiece in contact with the region of interest and storing the skin condition associated with the measurement position.

In one embodiment, the method may further include adjusting the direction and the distance of movement.

In one embodiment, the skin condition may be measured continuously or discontinuously.

In one embodiment, the skin condition may include a skin thickness and a skin elasticity of the region of interest.

In one embodiment, the skin condition may be associated with the measurement position for each point.

In one embodiment, the skin condition is associated with a region having the skin condition that is the same or falls within an allowable error range.

In one embodiment, the control parameter is a combination of a selection of the treatment medium to be applied to a treatment point corresponding to the treatment location, an intensity of the selected treatment medium, an output time of the selected medium, and an application depth of the selected medium.

According to the present invention, a composite treatment is possible using high-intensity focused ultrasound and laser according to the skin condition of the patient.

### BRIEF DESCRIPTION OF ACCOMPANYING DRAWINGS

Hereinafter, embodiments of the invention will be described with reference to the accompanying drawings. For the purpose of easy understanding of the invention, the same elements will be referred to by the same reference signs. Configurations illustrated in the drawings are examples for describing the invention, and do not restrict the scope of the invention. Particularly, in the drawings, some elements are slightly exaggerated for the purpose of easy understanding of the invention. Since the drawings are used to easily understand the invention, it should be noted that widths, thicknesses, and the like of elements illustrated in the drawings might change at the time of actual implementation thereof.

FIG. 1 exemplarily illustrates a skin treatment performed by use of a composite medical treatment apparatus;

FIG. 2A, FIG. 2B and FIG. 2C exemplarily illustrate the HIFU-laser generator of the composite medical treatment apparatus;

FIG. 3A and FIG. 3B exemplarily illustrate configurations of the composite medical treatment apparatus for measuring skin elasticity;

FIG. 4 is a flowchart exemplarily illustrating a method of driving the composite medical treatment apparatus using the skin map

FIG. 5A and FIG. 5B exemplarily illustrate a process of generating the skin map; and

FIG. 6 exemplarily illustrates the skin map and a treatment process using the same.

### DETAILED DESCRIPTION

Embodiments which will be described below with reference to the accompanying drawings can be implemented singly or in combination with other embodiments. But this is not intended to limit the present invention to a certain embodiment, and it should be understood that all changes, modifications, equivalents or replacements within the spirits and scope of the present invention are included. Especially, any of functions, features, and/or embodiments can be implemented independently or jointly with other embodiments. Accordingly, it should be noted that the scope of the invention is not limited to the embodiments illustrated in the accompanying drawings.

On the other hand, among terms used in this specification, terms such as “substantially,” “almost,” and “about” are used to take consideration of a margin or an error at the time of actual embodiment. For example, “substantially 90 degrees” should be construed to include angles at which the same advantages as at 90 degrees can be expected. For example, “almost zero” should be construed to include a quantity which is slightly present but is ignorable.

In the accompanying drawings, the same or similar elements will be referred to by the same reference numerals.

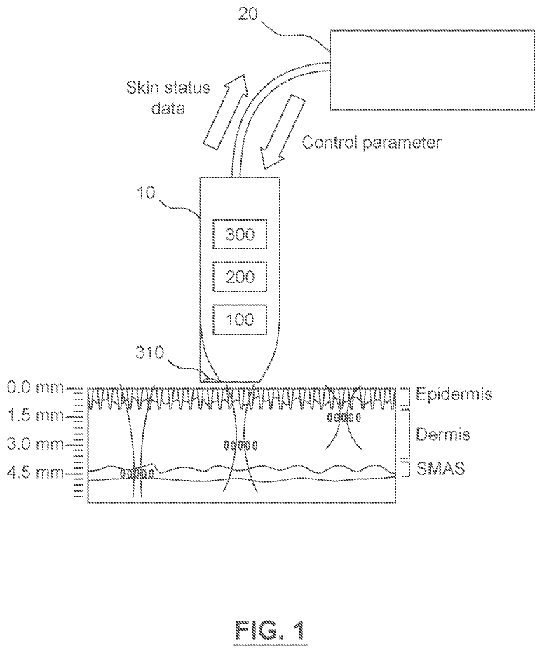

FIG. 1 exemplarily illustrates a skin treatment performed by use of a composite medical treatment apparatus.

The composite medical treatment apparatus may include a handpiece 10 and a main controller 20. The handpiece 10 collects information regarding skin condition, and generates and applies HIFU and/or laser to the skin according to the skin condition of the patient. The main controller 20 drives an ultrasonic source and a laser embedded in the handpiece 10 according to the collected skin condition.

The handpiece 10 measures the skin condition of the patient. In addition, the handpiece 10 generates high-intensity focused ultrasound and laser to be applied to the region of interest according to the control parameter determined by the measured skin condition. To this end, the handpiece 10 includes a HIFU-laser generator 100 that generates HIFU and laser, a skin elasticity measuring unit 200 that measures the elasticity of the patient's skin, and a position measuring unit 300 for measuring the position of the handpiece 10. Additionally, the handpiece 10 may measure the skin thickness of the region of interest.

The HIFU-laser generator 100 may output HIFU and/or laser according to the control parameter. The control parameters are, for example, information regarding a selection of the treatment medium, an intensity of the selected treatment medium, and an output time of the selected treatment medium. The HIFU-laser generator 100 may irradiate HIFU energy to a predetermined depth under the epidermis. In addition, the HIFU-laser generator 100 may irradiate the laser to the epidermis.

HIFU can remove the tissue by heating the body tissue to a temperature higher than body temperature (tissue heating) to induce necrosis of cells, or by exposing it to a high temperature for a short time (ablative therapy). Using HIFU, the temperature of a tissue, for example, a skin tissue, can be increased within a few seconds, and a part of the tissue can be necrotic through exposure at a temperature of about 55 degrees or higher for a certain period of time. The heat generated by ultrasonic waves appears differently depending on the energy absorption rate of the body tissue and the rate of heat diffusion and convection. In addition, the rate of increase in temperature varies depending on the frequency and continuity generated by the HIFU-laser generator 100, the period of the pulse wave, and the voltage between the peaks. By using these differences, the temperature change according to the target can be appropriately adjusted. Various treatments are also being attempted. In addition to the method of directly raising the temperature of the tissue, it also causes cellular changes according to changes in the living environment by changing the temperature by targeting surrounding tissues near the tissue. For example, as the permeability of capillaries increases or the lipid constituting the cell membrane becomes fluid due to the increase in temperature, the sensitivity of the cell to the external environment increases or the structure is destroyed. In addition, HIFU energy can also destroy fat cells. HIFU energy can be converted into thermal energy, and coagulative necrosis may occur in adipose tissue. Due to this, adipose tissue can be destroyed, and collagen can be is also contracted. The destroyed cells may cause a wound healing response, and when macrophages are generated, an inflammatory response may be produced, and macrophages can reduce adipose tissue by processing Lipids and cellular debris in the liver through the lymphatic system.

The skin tissue is composed of, for example, an epidermal layer, a dermal layer, and a SMAS layer. The HIFU-laser generator 100 may adjust the focal depth of HIFU energy. For example, when HIFU energy selectively coagulates the tissues in the dermal layer and the SMAS layer, it may cause the regeneration of collagen and elastic fibers in the skin tissue to obtain a skin lifting and/or tightening effect. Since the thickness or elasticity of the skin may be different for each patient and/or each region, and the depth of the target tissue may be different, HIFU energy may be controlled to have a different focal depth for each body part. Conventional HIFU apparatus may include cartridges detachable to the handpiece, each cartridge has a different focal depth. Therefore, the treatment had to be performed while replacing the cartridge according to the skin condition and/or location.

The skin elasticity measuring unit 200 measures the skin elasticity of the region of interest. The skin elasticity measurement is associated with the measurement position together with the skin thickness measurement. The skin condition may include the skin elasticity and the skin thickness associated with the measurement position. The skin elasticity measuring unit 200 may measure the skin elasticity of the region of interest in various ways. For example, the skin elasticity may be measured by using the time required for the deformed skin to be recoverd after applying pressure to the skin. The skin may be pressed to a certain depth by pressure or may protrude conversely.

The position measuring unit 300 measures a position of the point where the handpiece 10 came into contact with the region of interest for measurement of skin condition (hereinafter, measurement position) and a position of the point where the handpiece 10 comes into contact with the region of interest for treatment (hereinafter, treatment position) to generate two-dimensional or three-dimensional position information. The position measuring unit 300 receives a sensing information from an optical position sensor 310 that measures the distance and direction of movement when the handpiece 10 moves on the skin. In order to adjust the sensing information, the position measuring unit 300 may further include a sensor such as a gyro sensor (not shown) for measuring a posture of the handpiece 10, for example, rotation, inclination, and so on. Depending on a way of gripping the handpiece 10, sensing information that fails to accurately indicate the actual movement direction of the handpiece 10 may be generated.

The main controller 20 drives the handpiece 10 to collect the skin condition, generates a skin map based on the collected skin condition and measurement position, and generates control parameters to drive HIFU and/or laser according to the treatment position. The main controller 20 may include a storage unit for storing the skin map, a calculation unit for generating control parameters based on the skin condition corresponding to treatment position, a laser generator for generating laser to be applied to the region of interest through the handpiece 10, and a power supply for supplying power required to drive the composite medical treatment apparatus including the handpiece 10. The laser is delivered to the handpiece 10 through an optical fiber, and may be applied to the region of interest through a laser applicator in the handpiece 10.

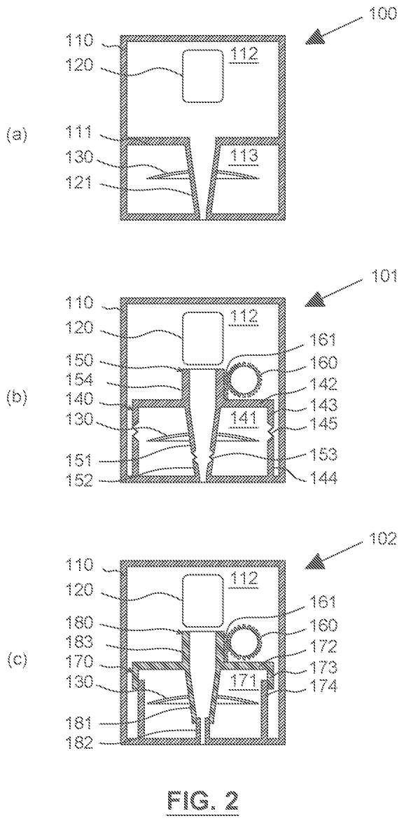

FIG. 2A, FIG. 2B and FIG. 2C exemplarily illustrate the HIFU-laser generator of the composite medical treatment apparatus, FIG. 2A shows the HIFU-laser generator with an ultrasonic transducer having a fixed focal length, FIG. 2B and FIG. 2C show the HIFU-laser generator with an ultrasonic transducer having a variable focal length.

Referring to FIG. 2A, the HIFU-laser generator 100 may include the laser applicator 120 and a housing 110 accommodating the ultrasonic transducer 130 therein. The housing 110 may be divided into an upper compartment 112 and a lower compartment 113 by an inner wall 111 extending in the lateral direction. A through hole to which a laser tube 121 is coupled is formed in the inner wall 111. At least a portion of a bottom of the housing 110 may be formed of an acoustically transparent material through which ultrasonic waves can pass. Meanwhile, at least a portion of the bottom may be formed of an optically transparent material through which the laser can pass. The laser applicator 120 is disposed in the upper compartment 112 to correspond to the through hole to which the laser tube 121 is coupled, and the ultrasonic transducer 130 is disposed in the lower compartment 113. The laser applicator 120 may be a collimating lens optically coupled to the optical fiber. The laser tube 121 extends in a downward direction from the inner wall 111 to the bottom of the housing 110. The lower compartment 113 accommodates the ultrasound transducer 130 and an ultrasonic medium that facilitates a transmission of HIFU. The laser tube 121 prevents the laser from being affected by the ultrasonic medium. The lower compartment 113 is isolated from the upper compartment 112 by the inner wall 111 and the laser tube 121.

The ultrasound transducer 130 may be a dual-mode ultrasound transducer. That is, the ultrasound transducer 130 may generate HIFU for the purpose of treatment and a low-intensity ultrasound for the purpose of measuring skin thickness. The ultrasonic transducer 130 may be fixed to an outer surface of the laser tube 121. The ultrasound transducer 130 may radiate HIFU or the low-intensity ultrasound for skin thickness measurement toward the bottom of the housing 110.

Referring to FIG. 2B, the HIFU-laser generator 101 may include the first housing 110 for accommodating the laser applicator 120 therein and the second housing 140 for accommodating the ultrasonic transducer 130 therein. The second housing 140 is disposed inside the first housing 110 and may share a bottom. The laser applicator 120 may be disposed on or over the second housing 140.

The second housing 140 may include a lower compartment 141 formed to be isolated from the upper compartment 112 by an upper wall 142 and lateral walls 143, 144 and 145. In the lower compartment 141, the ultrasonic transducer 130 and the ultrasonic medium are accommodated. The laser tube 150 extends in the downward direction from the upper wall 142 of the second housing 140 to the bottom of the first housing 110. The upper wall 142 of the second housing 140 includes a through hole through which the laser tube 150 is coupled. The lateral wall of the second housing 140 includes an upper lateral wall 143 extending in the downward direction from the upper wall 142, a lower lateral wall 144 extending in an upward direction from the bottom of the first housing 110, and a first stretchable connector 145 for coupling the upper lateral wall 143 and the lower lateral wall 144. The first stretchable connector 145 may have a bellows structure that may be expanded or contracted in the downward direction by a predetermined length.

The laser tube 150 may be also expanded and contracted in the downward direction. The laser tube 150 includes an upper laser tube 151, a lower laser tube 152, and a second stretchable connector 153. The upper laser tube 151 extends in the downward direction from the through hole formed in the upper wall 142 of the second housing 140 to the lower compartment 141, and the lower laser tube 152 extends in the upward direction from the bottom of the first housing 110 to the lower compartment 141. The second stretchable connector 153 expanding and contracting in the downward direction by a predetermined length couples the upper laser tube 151 and the lower laser tube 152.

The above-described HIFU-laser generator 100 may move the ultrasonic transducer 130 in the downward direction by a predetermined distance. The downward movement can be implemented by various structures, and FIG. 2B illustrates a structure with a rack-pinion. A rack 161 is attached or formed on an outer surface of a vertical extension 154 extending in the downward direction from the through hole formed in the upper wall 142 of the second housing 140 to the laser applicator 120. A pinion 160 engaged with the rack 161 is rotated clockwise/counterclockwise by a motor to move the ultrasonic transducer 130 in the downward direction by the predetermined distance. The ultrasonic transducer 130 is fixed to an outer surface of the upper laser tube 151, and the upper laser tube 151 is coupled to the upper wall 142 of the second housing 140. When the pinion 160 rotates counterclockwise, the ultrasonic transducer 130 coupled to the upper laser tube 151 moves in the downward direction, and on the contrary, when the pinion 160 rotates clockwise, the ultrasonic transducer 130 moves in the upward direction. When moving up and down, the first stretchable connector 145 and the second stretchable connector 153 may be expanded or contracted.

Referring to FIG. 2C, the HIFU-laser generator 102 may include the first housing 110 for accommodating the laser applicator 120 therein and a second housing 170 for accommodating the ultrasonic transducer 130 therein. The second housing 170 is disposed inside the first housing 110 and may share a bottom. The laser applicator 120 may be disposed on or over the second housing 170.

The second housing 170 may include an upper wall 172 that moves up and down, an upper lateral wall 173 extending in the downward direction from an edge of the upper wall, and a lower lateral wall 174 extending in the upward direction from the bottom of the first housing 110. The upper wall 172, the upper lateral wall 173, and the lower lateral wall 174 form a lower compartment 171 that is isolated from the upper compartment 112. In the lower compartment 171, the ultrasonic transducer 130 and the ultrasonic medium are accommodated. The laser tube 180 extends in the downward direction from the upper wall 172 of the second housing 170 to the lower compartment 171. The upper wall 172 of the second housing 170 includes a through hole through which the laser tube 180 is connected. The upper lateral wall 173 of the second housing 170 is disposed to overlap the lower lateral wall 174. As illustrated in FIG. 2C, an inner surface of the upper lateral wall 173 may be in contact with an outer surface of the lower lateral wall 174, or, conversely, the outer surface of the upper lateral wall 173 may be in contact with the inner surface of the lower lateral wall 174. Although not shown, a member such as O-ring for preventing an outflow of the ultrasonic medium an may be disposed on a location where the upper lateral wall 173 and the lower lateral wall 174 contact each other.

The laser tube 180 includes an upper laser tube 181 and a lower laser tube 182, and the upper laser tube 181 is disposed to overlap the lower laser tube 182. As illustrated in FIG. 2C, an inner surface of the upper laser tube 181 is in contact with an outer surface of the lower laser tube 182, or, conversely, the outer surface of the upper laser tube 181 is in contact with the inner surface of the lower laser tube 182. Although not shown, a member such as O-ring for preventing an outflow of the ultrasonic medium an may be disposed on a location where the upper laser tube 181 and the lower laser tube 182 contact each other.

The above-described HIFU-laser generator 102 may move the ultrasonic transducer 130 in the downward direction by a predetermined distance. The rack 161 is attached or formed on an outer surface of a vertical extension 183 extending in the downward direction from the through hole formed in the upper wall 172 of the second housing 170 to the laser applicator 120. The pinion 160 engaged with the rack 161 is rotated clockwise/counterclockwise by a motor to move the ultrasonic transducer 130 in the downward direction by the predetermined distance. The ultrasonic transducer 130 is fixed to an outer surface of the upper laser tube 181, and the upper laser tube 181 is coupled to the upper wall 172 of the second housing 170. When the pinion 160 rotates counterclockwise, the ultrasonic transducer 130 coupled to the upper laser tube 181 moves in the downward direction, and on the contrary, when the pinion 160 rotates clockwise, the ultrasonic transducer 130 moves in the upward direction. The maximum range of the vertical movement is a range in which the contact of the upper lateral wall 173 and the lower lateral wall 174 is maintained and the contact of the upper laser tube 181 and the lower laser tube 182 is maintained.

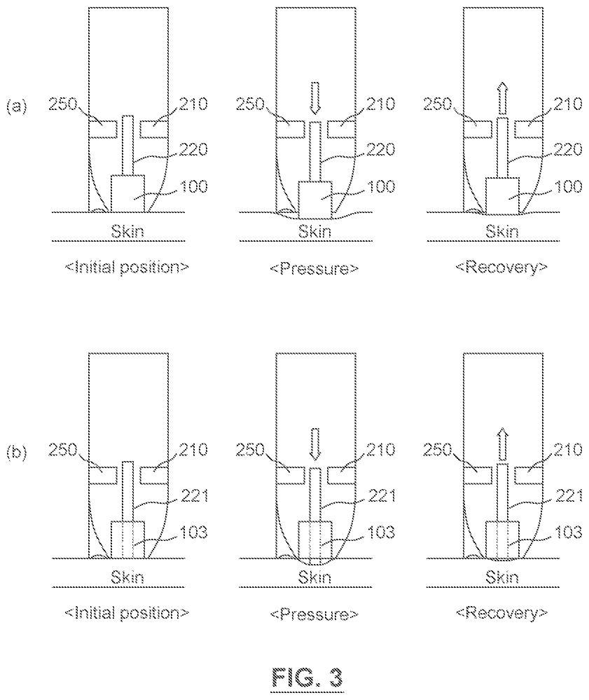

FIG. 3A and FIG. 3B exemplarily illustrate configurations of the composite medical treatment apparatus for measuring skin elasticity, (a) illustrates one configuration for measuring skin elasticity using a HIFU-laser generator, and (b) illustrates another configuration for measuring skin elasticity using a probe.

Skin elasticity may be measured by various ways such as measuring the time it takes for the skin to recover to its original state after it is deformed by applying pressure, measuring the pressure applied to the probe in contact with the pressed skin, or analyzing the moiré image obtained by photographing the skin. The method of measuring skin elasticity using the time taken to recover the original state will be described, but any other methods can be also used.

Referring to FIG. 3A and FIG. 3B, the handpiece 10 may measure a time or a physical quantity related to time (for example, an ascent distance within a reference time, a rate at which the skin rises, and so on. Hereinafter, collectively referred to as time) required to recover the original state after the skin is pressed inward by applying a certain pressure to the skin. The skin may be pressed by the HIFU-laser generator (100, 101, 102; hereinafter collectively referred to as 100) or the probe 221 for measurement. The handpiece 10 includes a vertical driver 210 for moving the HIFU-laser generator 100 or the measuring probe 221 in the downward direction and a displacement sensor 250 for measuring the rising distance. The vertical driver 210 such as an electromagnet generates an electromagnetic force corresponding to the applied electric signal to move the HIFU-laser generator 100 or the probe 221 in the downward direction. In the configuration illustrated in FIG. 3A, a vertical magnet bar 220 vertically coupled to the upper surface of the HIFU-laser generator 100 is shown to be moved in the downward direction by the vertical driver 210. However, this is only an example, and when a magnet is attached to a lateral surface of the HIFU-laser generator 100, the vertical driver 210 may move the HIFU-laser generator 100 directly in the downward direction. Meanwhile, in the configuration illustrated in FIG. 3B, although one probe 221 is exemplarily illustrated exemplified, but a plurality of probes may be equipped.

In an initial position, the HIFU-laser generator 100 or the probe 221 is located inside of the handpiece 10. In response to one control command input from the handpiece 10 or the main controller 20, the handpiece 10 applies a certain pressure to the skin. As illustrated, the vertical driver 210 may be moved in the downward direction by the HIFU-laser generator 100 or the probe 221 to press the skin to a preset depth. Thereafter, by another control command input from the handpiece 10 or the main controller 20, the handpiece 10 removes the pressure applied to the skin. For example, the vertical driver 210 stops pushing the HIFU-laser generator 100 or the probe 221 in the downward direction. Thereby, the skin will rise to its original state. As the skin rises to its original state, the HIFU-laser generator 100 or the measuring probe 221 also moves in the upward direction, and the displacement, that is, the vertical movement distance is measured by the displacement sensor 250. The measured displacement can be converted into skin elasticity by various conversion formulas.

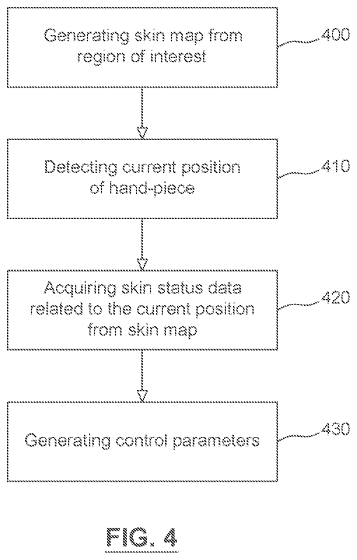

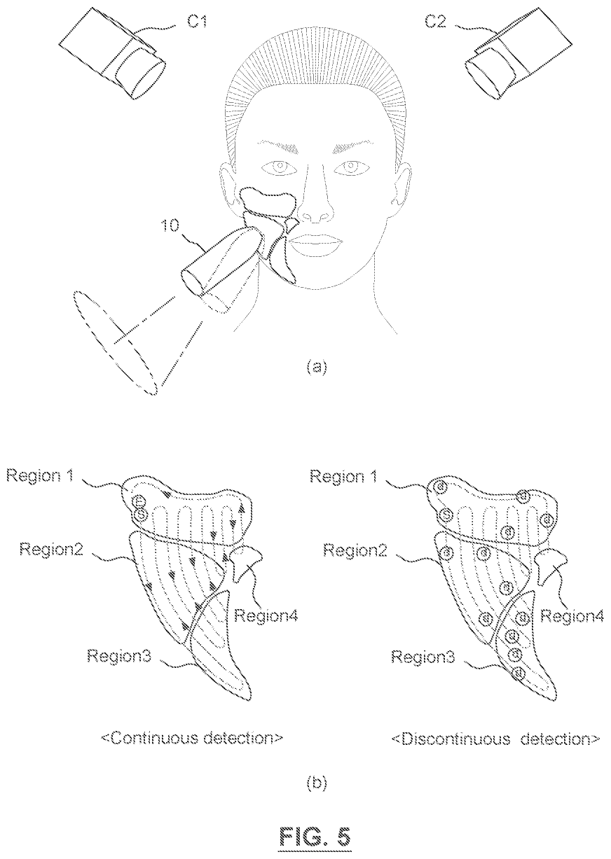

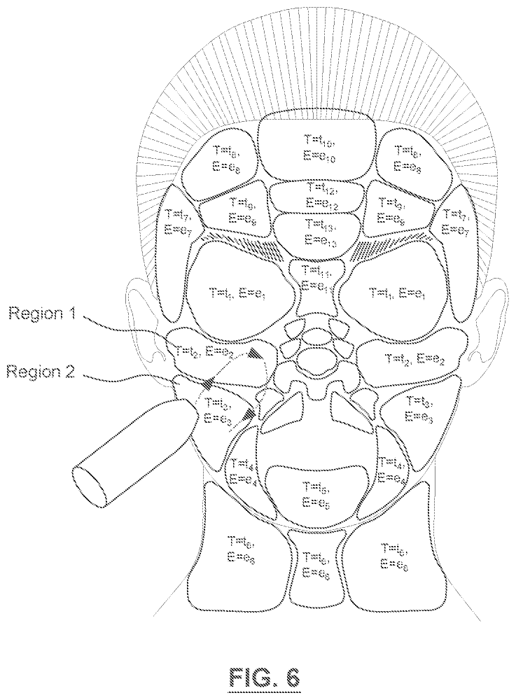

FIG. 4 is a flowchart exemplarily illustrating a method of driving the composite medical treatment apparatus using the skin map, FIG. 5A and FIG. 5B exemplarily illustrate a process of generating the skin map, and FIG. 6 exemplarily illustrates the skin map and a treatment process using the same.

Referring FIGS. 4 to 6 together, the skin map is generated using the skin condition measured at the region of interest 400. FIG. 5A exemplarily illustrates a process of measuring skin condition on a patient's face, and FIG. 5B exemplarily illustrates a process of measuring skin thickness and skin elasticity.

The measurement position may be generated by the handpiece 10 or by the three-dimensional scanner C1 or C2. The measurement position may be two-dimensional or three-dimensional coordinates for specifying a specific position over the entire region of interest, or a region identifier for specifying a plurality of sub-regions constituting the region of interest. Although there may be some discrepancies, for example, the region identifier may be used to classify and specify the cheeks into four regions 1 to 4 based on statistics on skin thickness.

The handpiece 10 may include the optical position sensor 310 and/or the gyro sensor, and may measure a direction and a distance of movement of the handpiece 10 in contact with the region of interest. The information measured by the sensor may be a position in the form of at least two-dimensional coordinates indicating a measurement point, and may be adjusted by information such as tilt and/or rotation measured by the gyro sensor. Additionally, when the handpiece 10 is moved to measure while maintaining a constant angle with the region of interest, the three-dimensional coordinates may be obtained using information such as an inclination measured by the gyro sensor. On the other hand, the three-dimensional scanner C1, C2 may perform a three-dimensional scan on the region of interest to generate three-dimensional coordinates and measure the measurement position of the handpiece 10. The measurement position generated by the handpiece 10 or the three-dimensional scanners C1 and C2 is used to generate the skin map.

Referring to FIG. 5B, the handpiece 10 may measure one or more skin condition while moving in contact with the region of interest. S indicates the position where the handpiece 10 started the measurement, the dotted line indicates a movement path of the handpiece 10, the arrow indicates the movement direction, d indicates the position where the skin condition is measured, and E indicates the position where the handpiece 10 finished the measurement. The skin condition may be continuously or discontinuously measured according to the characteristics of the corresponding information or the performance of the handpiece 10. For example, the skin thickness measurement using the ultrasonic transducer 130 may be performed continuously, whereas the skin elasticity measurement using the probe 221 may be performed discontinuously.

The skin condition is related to the measurement position. The skin condition may be measured while the handpiece acquires the measurement position. The skin condition may include at least the skin elasticity and the skin thickness related to the measurement position, and additionally include various information such as skin color, size/number of pores, moisture content and so on that can be specified for each patient/position of the region of interest. The skin condition may be stored for each point-by-point or sub-region. The skin condition may be stored in association with each specific point. The sub-region may be defined as a region having skin condition that is substantially the same or falls within an allowable error range in the region of interest, for example, a face. For example, the four regions 1 to 4 may be defined based on statistics on skin thickness, as described above, or may be defined by skin condition measured in the region of interest. In addition, the sub-region may be defined differently for each skin condition. For example, the sub-regions for each of the skin thickness and skin elasticity may be substantially the same, but may be defined at least partially differently.

The skin map is a set of one or more skin conditions related to measurement position. Two-dimensional or three-dimensional skin map may be displayed on a display. An example of the completed skin map is shown in FIG. 6. In addition to the above-described method of measuring skin condition related to the measurement position using the handpiece 10 or the three-dimensional scanner C1 or C2, the measurement position and the skin condition may be associated with each other in various ways. For example, if a specific position is displayed on the display while an image of the region of interest or a model depicting the region of interest is output to the display, the operator moves the handpiece 10 to the corresponding position to measure skin condition. The generated skin map may be used later in 410 to 430.

In the course of treatment, after the skin map is generated, when the handpiece 10 comes to be in contact with or is moved in contact with the region of interest, the treatment position is measured in 410. The treatment position may be measured in the same way as the measurement position.

One or more skin conditions associated with the treatment position is obtained in 420. The treatment position measured by the handpiece 10 is retrieved from the skin map stored in the main controller 20 or an external information processing device (e.g., PC or server; hereinafter referred to as the main controller 20 collectively) communicatively connected to the main controller 20. When the measurement position that matches the treatment position or falls within a predetermined error range is retrieved, one or more skin conditions related thereto may be acquired. If there is no measurement position matching the treatment position, the main controller 20 may calculate an average of one or more of skin conditions associated with measurement positions close to the contact position, or may select one or more skin condition related to the sub-region to which the treatment position belongs.

The control parameter is generated using the acquired one or more skin condition in 430. The control parameter is used to control selection of the treatment medium to be applied to the treatment point, the intensity of the selected treatment medium, an output time of the selected medium, an application depth where the selected medium can reach, and the like, based on one or more skin conditions. The treatment medium is, but is not limited to, HIFU and laser. For example, the main controller 20 may generate a control parameter for adjusting the focal depth of HIFU to be applied (e.g., the application depth of the selected medium) according to the skin thickness. The control parameter may be used to generate a control sequence for controlling two or more treatment mediums to operate simultaneously or sequentially.

The above description of the invention is exemplary, and those skilled in the art can understand that the invention can be modified in other forms without changing the technical concept or the essential feature of the invention. Therefore, it should be understood that the above-mentioned embodiments are exemplary in all respects, but are not definitive.

The scope of the invention is defined by the appended claims, not by the above detailed description, and it should be construed that all changes or modifications derived from the meanings and scope of the claims and equivalent concepts thereof are included in the scope of the invention.

What is claimed is:

1. A composite medical treatment apparatus using skin map, comprising:
a handpiece configured for measuring a skin condition associated with a measurement position on a region of interest in a skin map generation step, and configured for measuring a treatment position on the region of interest and applying a treatment medium according to a control parameter corresponding to the treatment position in a treatment step; and
a main controller configured for associating the measurement position with the skin condition in the skin map generation step, and configured for retrieving the skin condition associated with the measurement position based on the treatment position to generate the control parameter in the treatment step.

2. The composite medical treatment apparatus of claim 1, wherein the treatment medium is a high-intensity focused ultrasound and a laser,
wherein the handpiece comprises
an ultrasonic transducer configured for generating the high-intensity focused ultrasound and applying to the region of interest;
a laser applicator configured for applying the laser generated in the main controller to the region of interest; and
an optical position sensor configured for generating a measurement position and a treatment position information.

3. The composite medical treatment apparatus of claim 2, wherein the skin condition is a skin thickness,
wherein the ultrasound transducer is a dual-mode ultrasound transducer configured for applying a low-intensity ultrasound for measuring the skin thickness to the region of interest.

4. The composite medical treatment apparatus of claim 1, wherein the skin condition is a skin elasticity,
wherein the handpiece measures the skin elasticity from time to recover an original state after applying a pressure to the skin on the region of interest.

5. The composite medical treatment apparatus of claim 4, wherein the handpiece comprises
a probe being in contact with the skin;
a vertical driver configured for applying the pressure to the skin by moving the probe vertically, and removing the pressure when the probe comes into contact with the skin; and
a sensor configured for measuring a vertical displacement of the probe after the pressure is removed.

6. The composite medical treatment apparatus of claim 4, wherein the handpiece comprises
a HIFU-laser generator configured for generating the high-intensity focused ultrasound and the laser;
a vertical driver configured for applying the pressure to the skin by moving the HIFU-laser generator vertically, and removing the pressure when the HIFU-laser generator comes into contact with the skin; and
a sensor configured for measuring a vertical displacement of the HIFU-laser generator after the pressure is removed.

7. The composite medical treatment apparatus of claim 1, wherein the skin condition in the skin map is associated with the measurement position for each point.

8. The composite medical treatment apparatus of claim 1, wherein the skin condition in the skin map is associated with a region having the skin condition that is the same or falls within an allowable error range.

9. The composite medical treatment apparatus of claim 1, wherein the control parameter is a combination of a selection of the treatment medium to be applied to a treatment point corresponding to the treatment location, an intensity of the selected treatment medium, an output time of the selected medium, and an application depth of the selected medium.

10. A method of driving a composite medical treatment apparatus using a skin map, comprising:
generating a skin map by measuring a skin condition associated with a measurement position on a region of interest using a handpiece;
measuring a treatment position on the region of interest using the handpiece;
retrieving the skin condition associated with the measurement position corresponding to the treatment position from the skin map; and
generating a control parameter for controlling an operation of the composite medical treatment apparatus using the retrieved skin condition.

11. The method of claim 10, wherein the generating a skin map by measuring a skin condition associated with a measurement position on a region of interest using a handpiece comprises
measuring a direction and a distance of movement of the handpiece in contact with the region of interest;
measuring the skin condition of the region of interest while moving the handpiece in contact with the region of interest; and
storing the skin condition associated with the measurement position.

12. The method of claim 11 further comprising adjusting the direction and the distance of movement.

13. The method of claim 10, wherein the skin condition is measured continuously or discontinuously.

14. The method of claim 10, wherein the skin condition comprises a skin thickness and a skin elasticity of the region of interest.

15. The method of claim 10, wherein the skin condition is associated with the measurement position for each point.

16. The method of claim 10, wherein the skin condition is associated with a region having the skin condition that is the same or falls within an allowable error range.

17. The method of claim 10, wherein the control parameter is a combination of a selection of the treatment medium to be applied to a treatment point corresponding to the treatment location, an intensity of the selected treatment medium, an output time of the selected medium, and an application depth of the selected medium.
